(19) United States
(12) Patent Application Publication
(10) Pub. No.: US 2016/0030562 A1
(43) Pub. Date: Feb. 4, 2016

(54) METHODS TO ENHANCE CANCER TREATMENT

(71) Applicant: INTERNATIONAL BUSINESS MACHINES CORPORATION, Armonk, NY (US)

(72) Inventor: Eric Kline, Rochester, MN (US)

(21) Appl. No.: 14/883,931

(22) Filed: Oct. 15, 2015

Related U.S. Application Data
(62) Division of application No. 13/228,603, filed on Sep. 9, 2011.

(51) Int. Cl. A61K 39/395; A61K 45/06; A61N 5/10; C07K 16/30; G01N 33/68; A61K 31/40; A61K 47/48

(52) U.S. Cl. CPC A61K 39/39558; A61K 31/40; A61K 45/06; A61K 47/48584; C07K 16/3015; G01N 33/6854; A61N 5/10; A61K 2039/505

(57) ABSTRACT

Herein are provided methods for reducing or eliminating cancer in a patient in need of cancer treatment, by providing cholesterol deprivation therapy (CDT) in conjunction with antibodies directed against cholesterol-deprived tumor cells. Further provided are methods of enhancing the efficacy of other cancer treatments, by administering CDT and antibodies directed against cholesterol-deprived tumor cells, in combination with additional anti-cancer therapies.

### BACKGROUND OF THE DISCLOSURE

Cancer is a disease in which cumulative genetic mutations lead to uncontrolled cellular proliferation. In the United States alone, cancer causes the death of over a half-million people annually, with some 1.4 million new cases diagnosed per year. Cancer susceptibility is strongly associated with environmental factors, such as exposure to background radiation, smoking habits, and diet. Increased cholesterol levels, common in people eating a Western diet, have been linked to increased risk of several cancers, and also to resistance to chemotherapy (Montero, J., et al., Cancer Res 68:5246-56 (2008)).

Cholesterol, a specialized lipid molecule with a rigid four-ringed steroid structure and a hydrocarbon tail, is an integral component of eukaryotic cellular membranes. Cholesterol associates in the membrane with membrane proteins and with other lipid molecules, such as phospholipids. Cholesterol molecules orient themselves in the membrane with their polar head groups close to the polar head groups of membrane phospholipids and proteins. The inflexible steroid rings of a cholesterol molecule fill in spaces between phospholipids and “stiffen” the adjacent molecules. By decreasing the mobility of nearby protein and phosopholipid molecules, cholesterol makes the lipid bilayer less deformable, decreases potential for lipid-lipid and lipid-protein gap formation, and decreases the permeability of the membrane to small molecules (Alberts, B., et al., Molecular Biology of the Cell, 4^{th} Ed, New York: Garland Science; 2002). Cholesterol also positively associates with “lipid rafts”, specialized membrane areas that accumulate specific types of membrane proteins. Thus, cholesterol is an important component of cellular membranes.

Normal adults synthesize cholesterol at a rate of approximately 1 g/day and consume approximately 0.3 g/day. A relatively constant level of cholesterol in the body (150-200 mg/dL) is maintained in part by controlling the levels of de novo synthesis and dietary uptake. Cholesterol is utilized in the formation of membranes and in the synthesis of the steroid hormones and bile acids. Cholesterol synthesis occurs via the mevalonate pathway. There is a complex regulatory system to co-ordinate the biosynthesis of cholesterol with the availability of dietary cholesterol. The cellular supply of cholesterol is maintained at a steady level by regulation of a) the mevalonate pathway; b) intracellular free cholesterol; and c) plasma cholesterol levels.

Despite advances in medical treatment, cancer remains a major cause of death, and the primary forms of treatment for cancer, chemotherapy and radiation therapy, have significant side effects. New treatments that improve cancer outcomes, decrease cancer mortality rates and side effects from treatment, increase tumor cell sensitivity to treatment, and/or enhance existing cancer treatments, are sought.

### BRIEF SUMMARY OF THE DISCLOSURE

Herein are provided methods for reducing or eliminating cancer in a patient in need of cancer treatment, by providing cholesterol deprivation therapy (CDT) in conjunction with antibodies directed against cholesterol-deprived tumor cells. Further provided are methods of enhancing the efficacy of other cancer treatments, by administering CDT and antibodies directed against cholesterol-deprived tumor cells, in combination with additional anti-cancer therapies.

### DETAILED DESCRIPTION OF THE DISCLOSURE

Cholesterol and lipids in the cell membrane create the “structure” of the cellular surface. Membrane proteins are presented to the extracellular environment within the context of the surrounding lipid and cholesterol molecules. Cholesterol promotes cellular membrane rigidity and impermeability, and interacts with proteins in the lipid bilayer. The absence of necessary cholesterol can destabilize the lipid bilayer of the cellular membrane, creating increased susceptibility of the cell to the external environment.

Effects of cholesterol deprivation on cancer cells. As used herein, “cancer cells” are defined as cells exhibiting excessive proliferation and showing either potential to invade surrounding tissue, or actual invasion of surrounding tissue. In contrast, “non-cancerous cells” are normal, healthy cells that do not proliferate excessively and do not invade surrounding tissue. Cancer cells, which proliferate more rapidly than non-cancerous cells, have a greater need for cholesterol to create new cell membranes. Thus, cancer cells are more sensitive to cellular cholesterol availability. As cancer cells have greater susceptibility to cholesterol deprivation, the methods of the invention target cancer cells while having reduced or no effect on non-cancerous cells. The cancer cells that can be treated using the methods of the invention can be any type of cancer cell, including, but not limited to, melanoma, lymphoma, plasmacytoma, sarcoma, glioma, thymoma, leukemia, breast cancer, prostate cancer, colon cancer, esophageal cancer, brain cancer, lung cancer, ovarian cancer, cervical cancer or hepatoma.

Absence of bioavailable cholesterol can lead to production of cell membranes lacking adequate cholesterol, with associated lack of membrane rigidity, increased potential for micro-tears in the membrane, and greater tendency toward exposure of membrane protein regions, which would normally be hidden in the hydrophobic membrane interior, to the extracellular environment. In a membrane micro-tear, normally-hidden regions of membrane proteins, such as hydrophobic regions, can be exposed to the external environment. Exposure of such hydrophobic regions can present potentially antigenic epitopes to immune cells, such as B cells, dendritic cells, and T cells. Such exposed epitopes can then trigger an immune response, leading to an autoimmune reaction to the cancer cell.

Cancer cells differ from non-cancer cells in membrane protein expression profiles. For example, cancer cells have increased expression of MUC1, p53, and c-erbB2. Autoantibodies to these and other cancer-related proteins are evident in sera of cancer patients, yet the presence of these tumor antibodies does not lead to eradication of tumors expressing these proteins. Therefore, the invention provides anti-cancer cell antibodies generated outside of the patient, and administered back to the patient in a context that encourages a strong immune response to the patient's own cancer cells. The combination of altered membrane protein expression on cancer cell surfaces, cholesterol deprivation which leads to greater exposure of hidden epitopes and increased cancer cell vulnerability, and anti-cancer cell antibodies administered to the patient in an immune-sensitizing manner, provides the basis for an effective immune response to a patient's cancer. This is an effective cancer therapy that can improve patient outcomes when administered alone, and can also be effective in enhancing other cancer therapies when used in combination with, for example, chemotherapy, radiation therapy, and/or other cancer therapeutic strategies.

Reduction of cholesterol bioavailability can have multiple effects on the cellular environment, which can act synergistically with the immune response generated against cholesterol-deprived cancer cell protein epitopes to increase cancer cell damage. Cholesterol is an integral component of cellular membranes, so cholesterol deprivation can limit tumor growth by reducing the availability of cellular membrane material which is required for formation of new cells. This can reduce the rate of cellular proliferation. Another effect of cholesterol reduction can be decreased membrane rigidity that can reduce the potential for metastasis—which requires cell rigidity for the cell to become motile and traverse normal tissue boundaries. Further, the absence of membrane cholesterol can increase membrane permeability, which would increase the sensitivity of cancer cells to small molecule chemotherapeutic drugs and radiation therapy.

Cholesterol is also a precursor for synthesis of many hormones, such as androgens and estrogens. Thus, cholesterol deprivation can limit hormone synthesis, which can have beneficial anti-cancer effects, for example, if a patient has an estrogen-dependent breast cancer tumor that proliferates in an estrogenic environment, reduction in estrogen synthesis is desirable. Thus, cholesterol reduction can have the additional effect of limiting growth of hormone-influenced cancers, such as breast cancer and prostate cancer.

Thus, one embodiment herein provides a method to reduce or eliminate cancer in a patient, comprising administering cholesterol deprivation therapy to said patient. While the patient is undergoing cholesterol deprivation therapy, a sample of cancer cells is obtained from the patient, antibodies are generated against the sample of cholesterol-deprived cancer cells, and the patient is treated with said antibodies until the patient's cancer is reduced or eliminated.

Another embodiment provides a method to reduce or eliminate cancer in a patient, comprising obtaining a sample of cancer cells from said patient, then administering cholesterol deprivation therapy to said patient. While the patient is undergoing cholesterol deprivation therapy, the patient's cell sample is cultured with a cholesterol-reducing agent, antibodies are generated to the cholesterol-deprived cancer cells, and the patient is treated with said antibodies until the patient's cancer is reduced or eliminated.

Cholesterol deprivation therapy. Cholesterol deprivation therapy (“CDT”) is administered to a patient in need of treatment for cancer. Cholesterol is acquired for cellular needs through a combination of cellular de novo synthesis and diet. Therefore, CDT involves depriving the patient's system of cholesterol by a) reducing or eliminating dietary cholesterol intake and/or absorption, and b) partially or completely blocking de novo cholesterol synthesis.

Reducing or eliminating cholesterol intake and absorption. The primary means of reducing or eliminating dietary cholesterol occurs through changes in food intake. Total dietary cholesterol is to be limited to below 200 mg per day, preferably below 100 mg per day, even more preferably below 50 mg per day, most preferably dietary cholesterol intake is below 10 mg per day. A patient is assigned a diet (the “CDT diet”) in which high-cholesterol foods (such as egg yolks, high fat dairy products such as whole milk, organ meats, and pastries) and saturated fats are to be significantly reduced, and are preferably eliminated entirely. For example, a single whole egg contains 212 mg cholesterol, so whole eggs and egg yolks should be eliminated from the diet. Saturated fatty acids are precursors for cholesterol synthesis and therefore, the level of their intake is positively associated with higher cholesterol level. Major food sources of saturated fat are animal food-based products (bacon, lard, butter, etc.) and fried foods. Preferably, the patient limits intake of meat and animal products to one serving per day, for example, one serving of beef, pork, chicken, or fish, or one tablespoon of butter, each day.

It is recommended that the patient substitute unsaturated fats for saturated fats. Replacement of foods high in saturated fatty acids with polyunsaturated or monounsaturated fat rich foods reduces serum cholesterol levels. Unsaturated fat includes polyunsaturated fat and monounsaturated fat, both of which are predominantly found in plant products. Examples of polyunsaturated fat food sources include soybean, sunflower, fish and corn oils. Monounsaturated fat is found in high content in olive, peanut, and canola oils.

A CDT diet embraces low cholesterol, high fiber foods (such as unprocessed whole grains, vegetables, fruits, and proteins such as low-cholesterol fish and skinless chicken) as the primary food sources in the patient's diet. Foods rich in fiber, both soluble and insoluble fiber, prevent the re-absorption of cholesterol-rich bile acids from the small intestines back into circulation, thereby reducing circulating cholesterol. Thus, high-fiber, unprocessed foods inhibit cholesterol absorption and lower cholesterol in the body. The patient may also consume fiber supplements, such as Metamucil™ or Benefiber™, to increase dietary fiber intake.

Many of the foods emphasized in the CDT diet contain phytosterols, which reduce intestinal cholesterol absorption. Phytosterols are sterol compounds produced by plants which, because they are structurally very similar to cholesterol, inhibit cholesterol uptake in the digestive system. Phytosterols include plant sterols, esters of plant sterols, plant stanols or stanol esters and stanols and stanol esters derivable from plant sterols. Examples include alpha sitosterol, beta sitosterol, stigmasterol, ergosterol, campesterol, their fatty acid esters, and the like. Important sources of phytosterols are rice bran, corn bran, corn germ, wheat germ oil, corn oil, safflower oil, olive oil, cotton seed oil, soybean oil, e.g., soybean oil distillates, peanut oil, black tea, orange juice, green tea, kale, broccoli, sesame seeds, shea oils, grapeseed oil, rapeseed oil, linseed oil, and canola oil.

To minimize cholesterol intake, a vegetarian diet is preferred. A vegetarian diet avoids all animal meats, including beef, pork, chicken and fish; vegetable proteins (including nuts, soy-based products such as tofu, and beans and legumes in combination with rice) replace meat. Dairy products and eggs may or may not be included in a vegetarian diet. An ovo-vegetarian diet includes eggs but not dairy products, a lacto-vegetarian diet includes dairy products but not eggs, and an ovo-lacto vegetarian diet includes both eggs and dairy products. In order to limit cholesterol intake in a vegetarian diet that includes eggs and/or dairy, egg yolk and high fat dairy should be omitted. Egg whites and egg substitutes, and/or non-fat dairy products may be included. A multivitamin is recommended to make up for any dietary deficiencies, such as to provide adequate vitamin D (which is synthesized in the body from cholesterol).

A vegan diet is even more preferred. A vegan diet excludes animal products of any form; no meat (including chicken and fish); no eggs, no dairy, and no animal by-products (such as gelatin or honey). Vegetable proteins, vegetable-derived oils, fruits, vegetables, and grains are primary, preferably sole, food sources. Again, a multivitamin is recommended to make up for any dietary deficiencies, such as to provide adequate vitamin D and calcium (the majority of which is normally provided in a non-vegan diet by dairy products).

Prevention of dietary cholesterol absorption in the intestines may be augmented by treating the patient with a cholesterol absorption inhibitor (CAI) composition. CAIs include, for example, ezetimibe; 1,4-Diphenylazetidin-2-ones; 4-biarylyl-1-phenylazetidin-2-ones; 4-(hydroxyphenyl)azetidin-2-ones; 1,4-diphenyl-3-hydroxyalkyl-2-azetidinones; 4-biphenyl-1-phenylazetidin-2-ones; 4-biarylyl-1-phenylazetidin-2-ones; and 4-biphenylylazetidinones.

Another class of CAIs are bile acid sequestrants. Bile acid sequestrants reduce terminal ileal bile acid absorption, and thus also reduce cholesterol re-uptake in the intestines. Examples of bile acid sequestrants include cholestyramine, colesevelam and colestipol.

Blocking de novo cholesterol synthesis. In a second aspect, the cholesterol deprivation protocol involves partially or completely blocking de novo cholesterol synthesis by administering one or more cholesterol reducing agents to a patient. Cholesterol reducing agents encompass several classes of drugs that include HMG CoA reductase inhibitors (statins), γ-tocotrienol, bisphosphonates, cholesterol-ester-transfer-protein (“CETP”) inhibitors, squalene synthase inhibitors, soluble guanylate cyclase modulators (“sGC modulators”), nicotinic acid, and derivatives thereof (e.g. AGI-1067). In a preferred embodiment, the cholesterol reducing agent is a statin.

Statins, γ-tocotrienol, and bisphosphonates inhibit the mevalonate to cholesterol conversion pathway. Statins and γ-tocotrienol inhibit HMG-CoA reductase, a rate-limiting enzyme necessary for cholesterol production, and decrease the production of mevalonate and subsequent products on the way to construction of the cholesterol molecule. Statin therapy has been demonstrated to provide significant reductions in serum cholesterol levels. For example, administration of atorvastatin 80 mg daily significantly lowers plasma cholesterol concentrations. Statins include, but are not limited to, atorvastatin (Lipitor®), bervastatin, carvastatin, crilvastatin, dalvastatin, fluvastatin (Lescol®), glenvastatin, fluindostatin, velostatin, lovastatin (mevinolin; Mevacor®), pravastatin (Pravachol®), rosuvastatin (Crestor®), and simvastatin (Zocor®). Statins identical to lovastatin and its derivatives can be produced by a variety of filamentous fungi, including Monascus, Aspergillus, Penicillium, Pleurotus, Pythium, Hypomyces, Paelicilomyces, Eupenicillium, and Doratomyces [Manzoni M, Rollini M., Appl Microbiol Biotechnol. 58:555-64, 2002].

Bisphosphonates (such as clodronate and etidronate) that closely resemble pyrophosphate—a normal byproduct of human metabolism—are incorporated into adenosine triphosphate (ATP) analogues. The newest generation of bisphosphonates, which contain nitrogen (such as pamidronate, alendronate, risedronate, and ibandronate), are believed to inhibit post-translational modification within the mevalonate pathway.

A cholesterol reducing agent can be orally administered in the form of a sublingual tablet, buccal tablet, extended-release (long-acting) capsule, or spray. For a statin, about 2 mg to 80 mg, about 5 mg to 40 mg, or about 10 to 80 mg of a statin per day for an adult can be orally administered. For a cholesterol absorption inhibitor (e.g. ezetimibe), about 2 mg to 80 mg, about 5 mg to 40 mg, or about 10 to 80 mg of a cholesterol absorption inhibitor per day for an adult can be orally administered. For a bile acid sequestrant (e.g. cholestyramine, colesevelam or colestipol), about 1 g to 30 g, about 0.2 g to 6 g, about 0.1 g to 3 g, about 0.02 g to 0.6 g, about 0.01 g to 0.3 g, about 5 g to 150 g, about 2 g to 60 g or about 10 g to 300 g of a bile acid sequestrant per day for an adult can be orally administered.

Antibody therapy. In one embodiment of the invention, antibodies are generated against a sample of the patient's cancer cells which have been cholesterol-deprived (“CD cancer cells”). The patient's cancer cells may be deprived of cholesterol in vivo, by removing one or more samples of cells from the patient after the patient has been on CDT for a period of time, such as 5 to 60 days of CDT, 61 to 90 days of CDT, or more than three months of CDT. Samples of cells are removed from a patient by biopsy or other methods, such as blood samples, swabs, etc.

Alternatively, a sample of cells may be removed prior to administration of CDT to the patient, and the sample may be treated with one or more lipid altering agents, such as a statin, during in vitro culture of the cells in serum-free media, to prevent cholesterol production and uptake. Additionally, a cell sample removed while the patient has been on CDT for a period of time may be further treated with lipid altering agents during in vitro culture of the cell sample in serum-free media, to augment the cholesterol deprivation of these cells. Serum-free media and cell culture supplies may be obtained from, for example, Invitrogen, Sigma-Aldrich, and Irvine Scientific. Establishment of primary cell cultures from human tissue is well known in the art (see, for example, J. Davis, Basic Cell Culture: A Practical Approach, 2^{nd} Ed., Oxford University Press, 2002).

In a preferred embodiment, a sample of the patient's non-cancerous cells is removed in addition to a sample of the patient's cancer cells. For example, for a patient with a colorectal tumor, a sample of non-cancerous colorectal cells would be collected along with a sample of cancer cells during a biopsy of the patient's colorectal tumor. These non-cancerous cells would be cholesterol-deprived in an equivalent manner to the cancer cells. This sample of non-cancerous cells would be used to test the specificity of the antibodies to react to the patient's cancer cells but not react to the patient's non-cancerous, healthy cells, as described below.

Generation of anti-CD cancer cell antibodies. Cell samples isolated from a patient are used to generate antibodies to antigenic epitopes, revealed by cholesterol deprivation, that are present on the patient's cancer cells but absent or hidden on the patient's cholesterol-deprived non-cancerous cells. An “antibody” can be naturally occurring or man-made. Antibodies of the invention comprise monoclonal and polyclonal antibodies as well as fragments comprising the epitope-binding domain. An “anti-CD cancer cell antibody” is an antibody generated against a cholesterol-deprived (“CD”) cancer cell.

An “epitope” or “antigenic region”, as used herein, is a fragment of a protein that is immunologically reactive (i.e., specifically binds) with the B-cells and/or T-cell surface antigen receptors that recognize the protein. As used herein, antisera and antibodies are “CD cancer cell-reactive” or “CD cancer cell-specific” if they specifically bind to a cholesterol-deprived cancer cell (i.e., they bind to CD cancer cells, but show reduced or no binding with non-cancerous cells regardless of whether the non-cancerous cells are cholesterol-deprived).

In a preferred embodiment, antibodies generated against the patient's CD cancer cells are examined for cross-reactivity with the patient's CD non-cancerous cells. Antibodies that react equally with cancer cells and non-cancerous cells are considered non-specific for CD cancer cells, and are not preferred in the methods of the invention. Antibodies that react with CD cancer cells but show reduced or no reactivity to CD non-cancerous cells are considered specific to CD cancer cells, and are further utilized in the therapeutic methods of the invention.

Thus, in a preferred method, the step of obtaining a sample of cancer cells from a patient while said patient is undergoing CDT further comprises obtaining a second sample containing non-cancerous cells from said patient; and the antibodies generated against the cancer cells are tested for reactivity with said non-cancerous cells; wherein only antibodies with reduced reactivity to said patient's non-cancerous cells relative to reactivity to said patient's cancer cells are administered to said patient for treatment.

In another preferred method, the step of obtaining a sample of cancer cells from a patient further comprises obtaining a second sample containing non-cancerous cells from said patient; said non-cancerous cells are cultured with a cholesterol reducing agent in parallel with the culture of the cancer cells with said cholesterol reducing agent; and the antibodies generated against the cultured cancer cells are tested for reactivity with cultured non-cancerous cells; wherein only antibodies with reduced reactivity to said patient's non-cancerous cells relative to reactivity to said patient's cancer cells are administered to said patient for treatment.

Various methods for the preparation of antibodies are known in the art (see, Antibodies: A Laboratory Manual, CSH Press, Eds., Harlow, and Lane (1988); Harlow, Antibodies, Cold Spring Harbor Press, NY (1989)). For example, antibodies can be prepared by immunizing a suitable mammalian host with a sample of whole cells isolated from a patient. Briefly, such methods of generating an immune response (e.g. humoral and/or cell-mediated) in a mammal, comprise the steps of: exposing the mammal's immune system to a sample of CD cancer cells isolated from a patient, so that the mammal generates an immune response that is specific for CD cancer cells (e.g. generates antibodies that specifically recognize protein epitopes exposed by cholesterol deprivation).

Antibodies can be produced by cell culture techniques, including the generation of monoclonal antibodies as described herein, or via transfection of antibody genes into suitable bacterial or mammalian cell hosts, in order to allow for the production of recombinant antibodies. In one technique, a sample of cholesterol-deprived cells isolated from a patient is initially injected into any of a wide variety of mammals (e.g., mice, rats, rabbits, sheep or goats). A superior immune response may be elicited if the sample is injected along with a carrier protein, such as bovine serum albumin or keyhole limpet hemocyanin. The sample is injected into the animal host, preferably according to a predetermined schedule incorporating one or more booster immunizations, and the animals are bled periodically so that titers of antibodies can be taken to determine adequacy of antibody formation. Polyclonal antibodies specific for the polypeptide may then be purified from such antisera by, for example, affinity chromatography using cells from the patient sample coupled to a suitable solid support.

A monoclonal antibody is an antibody obtained from a population of substantially homogeneous antibodies, i.e., the antibodies comprising the population are identical except for possible naturally occurring mutations that are present in minor amounts. Monoclonal antibodies specific for protein epitopes exposed by cholesterol deprivation may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with CD cancer cells). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against protein epitopes exposed by cholesterol deprivation. Hybridomas having high reactivity and specificity for the patient's cancer cells over the patient's non-cancer cells are important for therapeutic purposes. When the appropriate immortalized cell culture is identified, the cells can be expanded and antibodies produced.

In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

The antibodies of the invention can also be produced by recombinant means. Antibodies that bind specifically to CD cancer cells can also be produced in the context of chimeric or complementarity-determining region grafted antibodies of multiple species origin. “Humanized” or human antibodies can also be produced, and are preferred for use in therapeutic contexts. Methods for humanizing murine and other non-human antibodies, by substituting one or more of the non-human antibody sequences for corresponding human antibody sequences, are well known (see for example, Jones et al., 1986, Nature 321: 522-525; Riechmann et al., 1988, Nature 332: 323-327; Verhoeyen et al., 1988, Science 239: 1534-1536, Carter et al., 1993, Proc. Natl. Acad. Sci. USA 89: 4285 and Sims et al., 1993, J. Immunol. 151: 2296). These humanized antibodies are designed to minimize unwanted immunological response toward rodent antihuman antibody molecules which limits the duration and effectiveness of therapeutic applications of those moieties in human recipients. Accordingly, preferred antibodies used in the therapeutic methods of the invention are those that are either fully human or humanized and that bind specifically to the patient's CD cancer cells with high affinity but exhibit low or no antigenicity in the patient.

Fully human monoclonal antibodies of the invention can be generated using cloning technologies employing large human Ig gene combinatorial libraries (i.e., phage display) (Griffiths and Hoogenboom, Building an in vitro immune system: human antibodies from phage display libraries. In: Protein Engineering of Antibody Molecules for Prophylactic and Therapeutic Applications in Man, Clark, M. (Ed.), Nottingham Academic, pp 45-64 (1993); Burton and Barbas, Human Antibodies from combinatorial libraries. Id., pp 65-82). Fully human monoclonal antibodies of the invention can also be produced using transgenic mice engineered to contain human immunoglobulin gene loci (see also, Jakobovits, 1998, Exp. Opin. Invest. Drugs 7(4): 607-614; U.S. Pat. No. 6,162,963 issued 19 Dec. 2000; U.S. Pat. No. 6,150,584 issued 12 Nov. 2000; and U.S. Pat. No. 6,114,598 issued 5 Sep. 2000).

Antibodies of the invention that treat cancers include those that initiate a potent immune response against the tumor or those that are directly cytotoxic. In this regard, antibodies of the invention can elicit tumor cell lysis by either complement-mediated or antibody-dependent cell cytotoxicity (ADCC) mechanisms, both of which require an intact Fc portion of the immunoglobulin molecule for interaction with effector cell Fc receptor sites on complement proteins. Mechanisms by which directly cytotoxic antibodies act include: inhibition of cell growth, modulation of cellular differentiation, modulation of tumor angiogenesis factor profiles, and the induction of apoptosis. The mechanism(s) by which a particular antibody of the invention exerts an anti-tumor effect can be evaluated using any number of in vitro assays that evaluate cell death such as ADCC, ADMMC, complement-mediated cell lysis, and so forth, as is generally known in the art.

Specificity of the anti-CD cancer cell antibody or antibodies can be tested by many techniques known in the art. For example, the specificity may be determined by ELISA. Whole cells isolated from the patient are used to coat the wells of a multi-well plate, using methods known in the art. Wells are coated with either CD cancer cells or CD non-cancerous cells. Anti-CD cancer cell antibodies are added, and reactivity with CD cancer cells relative to reactivity with CD non-cancerous cells is determined by antibody binding affinity. Other means of determining specificity, well known to those of skill in the art, include FACS analysis and immunochemistry.

Immunization. In therapeutic applications, anti-CD cancer cell antibodies are administered to a patient in an amount sufficient to elicit an effective B cell, CTL and/or HTL response to the patient's cancer cells and to cure or at least partially arrest or slow symptoms and/or complications. Amounts effective for this use will depend on, e.g., the particular composition administered, the manner of administration, the stage and severity of the disease, the weight and state of health of the patient, and the judgment of the prescribing physician.

Antibodies of the invention can be introduced into a patient such that the antibody binds to a CD cancer and mediates destruction and/or inhibits the growth of the tumor cells. Mechanisms by which such antibodies exert a therapeutic effect can include complement-mediated cytolysis, antibody-dependent cellular cytotoxicity, modulation of the physiological function of proteins of the invention, inhibition of ligand binding or signal transduction pathways, modulation of tumor cell differentiation, alteration of tumor angiogenesis factor profiles, and/or apoptosis. An immune response generated against CD cancer cells can lead to, for example, cancer cell death, or reduction in or prevention of, cancer cell proliferation.

In a preferred embodiment, one or more immunostimulants will be administered to the patient in addition to the anti-CD cancer cell antibody of this invention. An immunostimulant refers to any substance that enhances or potentiates an immune response (antibody and/or cell-mediated) to an antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Carriers that can be used with vaccines of the invention are well known in the art, and include, e.g., thyroglobulin, albumins such as human serum albumin, tetanus toxoid, polyamino acids such as poly L-lysine, poly L-glutamic acid, influenza, hepatitis B virus core protein, and the like. The vaccines can contain a physiologically tolerable (i.e., acceptable) diluent such as water, or saline, preferably phosphate buffered saline.

Upon administration of an antibody composition in accordance with the invention, via injection, aerosol, oral, transdermal, transmucosal, intrapleural, intrathecal, or other suitable routes, the immune system of the patient responds by producing large amounts of immune cells specific for the patient's cancer cells. Consequently, the patient becomes immune-sensitized to such cancer cells, or the patient derives at least some therapeutic benefit.

Further contemplated in this disclosure are methods of treating patients with the antibodies of the invention conjugated to a cytoxic agent. It is routine to conjugate antibodies to cytotoxic agents (see, e.g., Sievers et al. Blood 93:11 3678-3684 (Jun. 1, 1999)). When cytotoxic and/or therapeutic agents are delivered directly to cells, such as by conjugating them to antibodies specific for a molecule expressed by that cell, the cytotoxic agent will exert its known biological effect (i.e. cytotoxicity) on those cells. For example, antibodies can be conjugated to a toxin or radioisotope, such as the conjugation of calicheamicin or a maytansinoid or Y⁹¹ or I¹³¹ to an antibody.

Combination therapies. In a preferred embodiment, a patient is treated with anti-CD cancer cell antibodies in conjunction with other treatments, such as surgery, chemotherapeutic agents, androgen-blockers, immune modulators (e.g., IL-2, GM-CSF), and/or radiation therapy. Cancer immunotherapy using antibodies of the invention can be done in accordance with various approaches that have been successfully employed in the treatment of other types of cancer, including but not limited to colon cancer (Arlen et al., 1998, Crit. Rev Immunol. 18:133-138), multiple myeloma (Ozaki et al., 1997, Blood 90:3179-3186, Tsunenari et al., 1997, Blood 90:2437-2444), gastric cancer (Kasprzyk et al., 1992, Cancer Res. 52:2771-2776), B-cell lymphoma (Funakoshi et al., 1996, J. Immunother. Emphasis Tumor Immunol. 19:93-101), leukemia (Zhong et al., 1996, Leuk. Res. 20:581-589), colorectal cancer (Moun et al., 1994, Cancer Res. 54:6160-6166; Velders et al., 1995, Cancer Res. 55:4398-4403), and breast cancer (Shepard et al., 1991, J. Clin. Immunol. 11:117-127). To treat prostate cancer, for example, antibodies of the invention can be administered in conjunction with radiation, chemotherapy or hormone ablation.

Antibody therapy can be useful for all stages of cancer, including advanced or metastatic cancers. Treatment with the anti-CD cancer cell antibody therapy of the invention is indicated for patients who have received one or more rounds of chemotherapy. Alternatively, antibody therapy of the invention is combined with a chemotherapeutic or radiation regimen for patients who have not previously received chemotherapeutic treatment. Additionally, antibody therapy can enable the use of reduced dosages of concomitant chemotherapy, particularly for patients who do not tolerate the toxicity of the chemotherapeutic agent very well. Fan et al. (Cancer Res. 53:4637-4642, 1993), Prewett et al. (International J. of Onco. 9:217-224, 1996), and Hancock et al. (Cancer Res. 51:4575-4580, 1991) describe the use of various antibodies together with chemotherapeutic agents.

Therapeutic methods of the invention contemplate the administration of single antibodies as well as combinations, or cocktails, of different antibodies. Such antibody cocktails can have certain advantages inasmuch as they contain antibodies that target different epitopes, exploit different effector mechanisms or combine directly cytotoxic antibodies with antibodies that rely on immune effector functionality. Such antibodies in combination can exhibit synergistic therapeutic effects.

Antibody formulations of the invention are administered via any route capable of delivering the antibodies to a cancer cell. Routes of administration include, but are not limited to, intravenous, intraperitoneal, intramuscular, intratumor, intradermal, and the like. Treatment generally involves repeated administration of an antibody preparation of the invention, via an acceptable route of administration such as intravenous injection (IV), typically at a dose in the range of about 0.1, 0.2, 0.3, 0.4, 0.5, 0.6, 0.7, 0.8, 0.9, 1, 2, 3, 4, 5, 6, 7, 8, 9, 10, 15, 20, or 25 mg/kg body weight. In general, doses in the range of 10-1000 mg antibodies per week are effective and well tolerated.

Based on clinical experience with the Herceptin™ monoclonal antibody in the treatment of metastatic breast cancer, an initial antibody loading dose of approximately 4 mg/kg patient body weight IV, followed by weekly doses of about 2 mg/kg IV of the antibody preparation represents an acceptable dosing regimen. Preferably, the initial loading dose is administered as a 90 minute or longer infusion. The periodic maintenance dose is administered as a 30 minute or longer infusion, provided the initial dose was well tolerated. As appreciated by those of skill in the art, various factors can influence the ideal dose regimen in a particular case. Such factors include, for example, the binding affinity and half life of the antibody or antibodies used, the degree of expression of the protein of the invention in the patient, the extent of circulating shed CD cancer cell proteins, the desired steady-state antibody concentration level, frequency of treatment, and the influence of chemotherapeutic or other agents used in combination with the treatment method of the invention, as well as the health status of a particular patient.

Anti-idiotypic antibodies are also contemplated in the invention. Anti-idiotypic antibodies of the invention can be used to induce an immune response to CD cancer cells. The generation of anti-idiotypic antibodies is well known in the art; this methodology can readily be adapted to generate anti-idiotypic anti-protein of CD cancer cell antibodies that mimic a CD cancer cell protein epitope (see, for example, Wagner et al., 1997, Hybridoma 16:33-40; Foon et al., 1995, J. Clin. Invest. 96:334-342; Herlyn et al., 1996, Cancer Immunol. Immunother. 43:65-76). Anti-idiotypic antibodies can be used to further enhance cancer treatments as described herein.

Radiation/chemotherapy. The term “cancer therapeutic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to maytansinoids, yttrium, bismuth, ricin, ricin A-chain, doxorubicin, daunorubicin, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, sapaonaria officinalis inhibitor, and glucocorticoid and other chemotherapeutic agents, as well as radioisotopes.

As used herein, “treatment” refers to clinical intervention in an attempt to alter the disease course of the individual or cell being treated, and can be performed either for prophylaxis or during the course of clinical pathology. Therapeutic effects of treatment include without limitation, preventing occurrence or recurrence of disease, alleviation of symptoms, diminishment of any direct or indirect pathological consequences of the disease, decreasing the rate of disease progression, amelioration or palliation of the disease state, and remission or improved prognosis.

An effective amount is an amount sufficient to effect a beneficial or desired clinical result upon treatment. An effective amount can be administered to a patient in one or more doses. A therapeutically effective amount is an amount that is sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the-patient, the condition being treated, the severity of the condition and the form and effective concentration of the binding protein administered.

Patients undergoing CDT must be carefully monitored for side effects such as muscle weakness, liver dysfunction, dizziness, headaches, memory loss, digestive problems, depression, and low libido. The prescribing physician can modify the CDT and/or other therapies to minimize side effects while maximizing results.

### EXAMPLE 1

Patient A has Stage II breast cancer. Patient A is in year 3 of a 5-year treatment with tamoxifen (tamoxifen prescribed at 20 mg a day for 5 years) but no regression in patient's breast cancer is apparent. Additional treatment is desired.

A sample of breast cancer cells and non-cancerous breast cells are removed from Patient A by biopsy. Patient's cancer cells and non-cancer cells are cultured separately in serum-free defined DMEM/F12 media containing 10 mg/ml insulin and 10 ng/ml epidermal growth factor (EGF). Cells are cultured with 10 μM lovastatin as described in Wali, V. B., et al., Exp. Biol. Med. 234:639-650 (2009).

Cholesterol deprivation therapy is prescribed for Patient A. Patient A prefers a vegan diet, so Patient A's diet includes no meat or animal products, no saturated fats, and monounsaturated and polyunsaturated fats in moderation. Vegetable proteins, fruits, vegetables, and grains are primary food sources. A multivitamin with vitamin D and calcium is prescribed. Treatment with 80 mg/day atorvastatin (Lipitor®) is prescribed in concert with dietary modifications. CDT is prescribed for 8 months.

While Patient A follows CDT diet, Patient A's cultured CD cancer cells are injected into transgenic mice engineered to contain human immunoglobulin gene loci, and anti-CD cancer cell monoclonal antibodies are produced from immortalized hybridomas, as described in U.S. Pat. No. 6,150,584. These antibodies are tested by ELISA to determine specificity for the patient's CD cancer cells. Isolates of Patient A's cultured cells are used to coat the wells of a multi-well plate, using methods known in the art. Wells are either coated with CD cancer cells or CD non-cancerous cells. After one night of contact, the wells are washed 3 times with a solution of PBS/0.05% tween 20, then saturated with a solution of 0.1M Tris 20%, sucrose pH 7.8. Anti-CD cancer cell antibodies are added in duplicates, with 10-fold serial dilutions. After a 6 h incubation, the wells are washed three times with a solution of PBS 0.05%, Tween 20 and an anti-humanized mouse antibody coupled with peroxidase (Biosource) diluted to 1/5,000 in PBS, 0.1% BSA, 0.01% Tween 20 is added for an additional two hours. Binding of the antibody is revealed using ABTS [2,2′-azino-bis(3-ethylbenzthiazoline-6-sulfonic acid)] as a substrate, and the reading is carried out at 405 nm. The results show that the anti-CD cancer cell antibodies specifically bind CD cancer cells with high affinity, and show almost no reactivity with CD non-cancerous cells.

Hybridomas producing monoclonal antibodies with high specificity for Patient A's CD cancer cells, as defined by strong binding to Patient A's CD breast cancer cells but poor binding to Patient A's CD non-cancerous breast cells, are expanded and antibodies are produced for therapeutic treatment of Patient A.

Patient A is treated with anti-CD cancer cell antibodies. Initial antibody loading dose of 1.3 g antibody preparation is administered as a two hour IV infusion, followed by weekly doses of about 0.6 g administered as a 30 minute infusion of the antibody preparation. Antibody and CDT treatments continue for 6 months.

As a result of combination treatment with CDT diet, statins, anti-CD cancer cell antibodies, and tamoxifen, Patient A's breast cancer regresses.

What is claimed is:

1. A method to reduce the number of cancer cells in a patient, comprising:
a. Administering cholesterol deprivation therapy to said patient continuously through step (d);
b. Obtaining a sample of cancer cells from said patient, while said patient is undergoing cholesterol deprivation therapy, wherein said sample of CD cancer cells are cultured with a cholesterol reducing agent;
c. Generating CD cancer cell-specific antibodies that show reduced or no reactivity with said patient's CD non-cancerous cells; and
d. Treating said patient with an effective amount of said CD cancer cell-specific antibodies for a time sufficient to reduce cancer cells in said patient.

2. The method of claim 1, wherein step (b) further comprises obtaining a second sample containing non-cancerous cells from said patient; and step (c) further comprises testing reactivity of the generated antibodies with said non-cancerous cells; wherein only antibodies with reduced reactivity to said patient's non-cancerous cells relative to reactivity to said patient's cancer cells are administered to said patient in step (d).

3. The method of claim 1, wherein said cholesterol deprivation therapy comprises administering to said patient one or more cholesterol reducing agents while also eliminating or reducing cholesterol in said patient's diet.

4. The method of claim 3, wherein one of the one or more cholesterol reducing agents applied to said patient is a statin.

5. The method of claim 1, wherein step (d) further comprises treatment of said patient with radiation therapy.

6. A method to eliminate cancer in a patient, comprising:
a. Obtaining a sample of cancer cells from said patient;
b. Administering cholesterol deprivation therapy to said patient continuously through step (e);
c. Culturing said cancer cells with a cholesterol reducing agent;
d. Generating antibodies to said cancer cells; and
e. Treating said patient with said antibodies until said patient's cancer is reduced or eliminated.

7. The method of claim 6, wherein step (a) further comprises obtaining a second sample containing non-cancerous cells from said patient; step (c) further comprises culturing said non-cancerous cells with a cholesterol reducing agent; and step (d) further comprises testing reactivity of the generated antibodies with the cultured non-cancerous cells; wherein only antibodies with reduced reactivity to said patient's non-cancerous cells relative to reactivity to said patient's cancer cells are administered to said patient in step (e).

8. The method of claim 6, wherein said cholesterol deprivation therapy comprises administering to said patient one or more cholesterol reducing agents while also eliminating or reducing cholesterol in said patient's diet.

9. The method of claim 8, wherein one of the one or more cholesterol reducing agents applied to said patient and said cultured cells is a statin.

10. The method of claim 6, wherein step (e) further comprises treatment of said patient with radiation therapy or a chemotherapeutic agent.

11. The method of claim 1, wherein step (d) further comprises administration of said antibodies with one or more immunostimulants.

12. The method of claim 6, wherein step (e) further comprises administration of said antibodies with one or more immunostimulants.

13. The method of claim 1, wherein the antibodies generated in step (c) are humanized prior to treatment of said patient with said antibodies.

14. The method of claim 6, wherein the antibodies generated in step (d) are humanized prior to treatment of said patient with said antibodies.

15. The method of claim 1, wherein the antibodies generated in step (c) are conjugated to a cytotoxic agent prior to treatment of said patient with said antibodies.

16. The method of claim 6, wherein the antibodies generated in step (d) are conjugated to a cytotoxic agent prior to treatment of said patient with said antibodies.

17. The method of claim 1, wherein in step (b), said sample of CD cancer cells, and said sample of CD non-cancerous cells, are obtained from said patient after 5 to 60 days of cholesterol deprivation therapy.
